(19) United States
(12) Patent Application Publication
(10) Pub. No.: US 2009/0054993 A1
(43) Pub. Date: Feb. 26, 2009

(54) Coded trial neck components

(75) Inventors: Franck Le Bon, Evrecy (FR); Pascal Collet, Saint-Martin-De-Fontenay (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 12/214,420

(22) Filed: Jun. 18, 2008

(30) Foreign Application Priority Data
Aug. 22, 2007 (GB) 0716403.1

(51) Int. Cl. A61F 2/36; A61F 2/32

(52) U.S. Cl. 623/23.15; 623/22.11

(57) ABSTRACT

A trial prosthetic hip system has a plurality of trial stems each having a recessed bore in a proximal end thereof. At least three sets of trial necks are provided each having a connection element for inserting into the bore in the trial stems. A first set of trial necks having a valgus offset with one of the necks of the first set being anteverted, one be retroverted and one being neutral in version. A second set of trial necks is provided having a varus offset with one neck of the second set being anteverted, one being retroverted and one being neutral in version. A third set of trial necks is provided being neutral in varus-valgus, one neck of the third set being anteverted, one being retroverted and one being anteverted and each trial neck of the first, second and third sets having a coded marking on a proximally facing surface indicating the varus/valgus, anteverted/retroverted and neutral orientations.

### BACKGROUND OF THE INVENTION

This invention relates to a trial prosthetic neck component which is adapted for use with a trial femoral stem or broach or an implanted femoral stem.

The trial prosthetic neck component, according to the invention, is intended for use in a modular operative technique before fitting a femoral implant which employs a modular prosthetic neck component the distal end of which is attached to a femoral stem and the proximal end of which carries or is attached to a bearing head in the shape of a part-spherical ball. The use of the modular neck component enables the surgeon to provide any required offset to suit the joint at the time of implantation.

The anatomy of the femur varies considerably from one individual to another. Version of the proximal femur refers to the relationship of the axis of the femoral neck to the transcondylar axis of the distal femur. Femoral anteversion refers to the condition where the femoral neck axis is rotated anterior with respect to the transcondylar femoral axis, with the femoral head directed anterior to the coronal plane of the femur. In femoral retroversion, the femoral neck axis is oriented posterior to the transcondylar axis thus positioning the femoral neck and head posterior to the coronal plane of the femur. Femoral anteversion of 10° to 20° along with acetabular anteversion provides inherent stability to the hip joint.

Varus/valgus describe the alignment between the axis of the femoral shaft and the neck of the femur. To visualize the alignment, a line is drawn to illustrate the long axis of the proximal neck. Another line is drawn to illustrate the long axis the femur. We then compare the two lines, focusing on the distal segment's alignment with respect to the proximal segment. In a varus alignment, the distal segment deviates medially with respect to the proximal segment. In a valgus alignment, the distal segment deviates laterally with respect to the proximal segment.

As used herein when referring to bones or other parts of the body, the term “proximal” means close to the heart and the term “distal” means more distant from the heart. The term “inferior” means toward the feet and the term “superior” means toward the head. The term “anterior” means toward the front part or the face and the term “posterior” means toward the back of the body. The term “medial” means toward the midline of the body and the term “lateral” means away from the midline of the body.

### SUMMARY OF THE INVENTION

According to the present invention a trial prosthetic neck component adapted for use with a trial femoral stem or broach, or an implanted femoral stem, has a first connection element for removably connecting it, at its distal end, to the trial femoral steal or broach, or an implanted femoral stem. The neck component has a second connection element at its proximal end for connection to a bearing head, and in which the proximal end is provided with indicea to indicate its length and any valgus, varus, anteverted, retroverted offset.

Thus, a number of trial neck components, as set out above, can be provided and the operating surgeon can select an appropriate trial neck having the necessary offset and length to suit the patient. He can assess the neck component when fitted or carrying the bearing head and by selecting the appropriate component achieve the desired result for a good fit. The indicea provided on the end of the component enables the surgeon to then select the appropriate modular neck component from a supply and will enable the surgeon to build up the implant as required.

In a preferred embodiment the indicea indicating offset comprises a line extending in the direction of the offset. With this arrangement the indicea which indicates the length of the component can be provided by a colored dot the longer the length the greater the diameter of the dot. Each line indicating the offset preferably extends outwardly from the length indicating dot provided at a central point on the proximal end of the neck component. The line thus points in the direction of the offset and extends away from the length indicating dot.

The connection element which allows removable connection of the neck component to the trial stem or broach or the actual stem may include a cylindrical plug and an angularly located socket.

With this arrangement the cylindrical plug and angularly located socket can be adapted to engage and fit an existing socket and projecting abutment on the broach, and the connecting element also includes a connector the proximal end of which has a socket and projecting abutment to engage the plug and socket on the neck component and the distal end of which has a spade connector to engage and locate in the trial stem or implanted femoral stem.

With this arrangement the proximal end of the connector may include a plug and an angularly located socket and the distal end of the neck component can have a socket to receive the plug and a projecting abutment to engage the angularly located socket. This arrangement ensures that the connector is fixed against relative angular rotation to the stem when in position.

In order to provide a connection which can be a tight push fit the connector can be made of a synthetic plastics material.

The trial prosthetic neck component, according to the invention, can be provided in combination with a number of other similar trial neck components which have different lengths and/or offsets and which can be selectively connected to a trial medullary stem or broach or an implanted femoral stem and a bearing head to provide a modular construction of trial femoral implants of different lengths and offsets so that a surgeon can achieve the desired length and offset of either a trial implant provided by a trial stem or a broach or by fitting the trial neck component to a femoral stem which has already been implanted.

The trial prosthetic neck component can therefore be provided in combination with eight similar trial neck components, nine of the components having long necks, three of the long neck components having a valgus offset and one of which also has an anteverted offset, another of which also has a retroverted offset and a third of which has no anteverted or retroverted offset; another three of said long neck components having a varus offset one of which also has an anteverted offset another of which also has a retroverted offset and a third of which has no anteverted or retroverted offset; and the remaining three of said long neck components having no valgus or varus offset, one of said three having an anteverted offset, another having a retroverted offset and a third of which has no anteverted or retroverted offset; the remaining nine neck components having short necks and replicating the various offsets of the nine long neck components.

With this arrangement the operating surgeon is able to choose a trial neck component which is of long or short neck length and which has offsets in all the available directions. The indicea on the proximal end of the component which he/she has selected enables him/her to then take the appropriate modular component which is to be secured to the stem.

In a preferred arrangement each of the nine long neck trial components carries a colored dot to indicate the particular offset, each two components have offsets which are the reciprocal of each other and one of which has a valgus effect and the other a varus effect having the same color, and the components which do not have a valgus or varus effect having the same color; and the nine neck components having short necks and replicating the various offsets of the nine long neck components having a similar color coding. The colored dot which indicates the length of the component can also carry the color coding which indicates the offset.

Suitable colors are brown, green, blue, yellow and gray.

This arrangement enables the use of only ten modular necks for use in the implanted stem. Because they can be constructed so that in each of the constructions they can be used in the reciprocal directions merely by rotating them through 180°, thus a neck which has a varus anteverted offset can also be used by rotating it through 180° as a neck valgus and retroverted offset.

The modular necks which are to be inserted in the fixed stem also, preferably, carry the color coding of the test necks so that if, for example, the color of the neck which had varus and anteverted offset is yellow and can be used with an implanted stem could be used by the surgeon but he could also use the same yellow neck if the test neck indicated that an offset of varus retroverted was required merely by rotating the modular stem to the appropriate direction. Modular necks could also carry the direction indicators as used by the test necks to enable this to be done.

The invention also includes a trial prosthetic neck component as set forth above in combination with seventeen similar trial neck components which have the color coding and offset indicea form part of a kit of parts which also includes ten modular necks of different offsets which can be used when the required offset has been determined by the trial offset necks and one or more modular stems which were used to replace the trial broach of stem, together with one or more trial broaches or stems.

The kit of parts could also include one or more bearing heads attachable to the appropriate modular neck or the modular necks could carry integral bearing heads.

The spade is provided on the distal end of the connector. A similar spade-shape connector is provided on the modular necks but in this case is a tight or interference fit whereas the spade on the connector which is preferably made of a synthetic plastics material is a tight or push fit into the slot.

Various aspects of the invention are provided by a trial prosthetic hip system having a plurality of trial stems each having a recessed bore in a proximal end thereof and at least three sets of trial necks each having a connection element for inserting into the bore in the trial stems. A first set of trial necks has a valgus offset with one of the necks of the first set being anteverted, one be retroverted and one being neutral in version. A second set of trial necks is provided having a varus offset with one neck of the second set being anteverted, one being retroverted and one being neutral in version. A third set of trial necks is provided being neutral in varus/valgus, one neck of the third set being anteverted, one being retroverted and one being anteverted. Each trial neck of the first, second and third sets has a coded marking on a proximally facing surface indicating the varus/valgus, anteverted/retroverted and neutral orientations.

There are six sets of trial necks with three sets having short necks and three sets having long necks. The necks are coded to indicate which are short necks and which are long necks. The neck lengths are indicated by colored circular dots having a diameter proportioned to the neck length. The necks include a trunion for connection to a prosthetic femoral head, the trunion having a proximally facing surface including indicia of the varus/valgus, anteverted/retroverted and neutral orientation. The offsets are indicated by lines extending along a proximally facing surface of the trunion. The recessed bore in the trial stem preferably has a non-circular cross section and the connection element on the trial stem is an oval spade.

Each trial stem has a recessed bore in a proximal end thereof and a plurality of sets of trial necks are provided including sets having at least two different neck lengths and different varus/valgus and anteversion/retroversion orientations. Each trial neck includes a trunion for receiving a femoral head element. A proximally facing surface of the trunion including markings for indicating neck length, varus/valgus and anteversion/retroversion orientation. In the preferred embodiment there are six sets of trial necks with three sets having short necks and three sets having long necks. The necks are coded to indicate which are short necks and which are long necks. The neck lengths are preferably indicated by colored circular dots having a diameter proportioned to the neck length. The neck offsets are indicated by lines extending along a proximally facing surface of the trunion. The recessed bore in the trial stem has a non-circular cross section and the connection element on the trial stem is an oval spade.

### BRIEF DESCRIPTION OF THE DRAWINGS

The invention can be performed in various ways and one embodiment will now be described by way of example and with reference to the accompanying drawings in which

FIG. 1 is a diagrammatic isometric view showing the proximal end of a femur the head of which has been resected to receive an implant having a stem located within the femur and projecting neck to which a bearing, in the form of a hemispherical ball can be fitted;

FIG. 2 is an isometric view of a modular neck similar to that shown in FIG. 1 which can be located in an implanted stem;

FIG. 3 is a diagrammatic isometric view from one side of a trial prosthetic neck component, according to the invention;

FIG. 4 is an isometric view from the front of eighteen alternative trial neck components according to the invention;

FIG. 5 is a diagrammatic view of the proximal ends of the components shown in FIG. 4;

FIG. 6 shows left and right handed prosthetic stems which can be used with the present invention; and

FIG. 7 is a diagrammatic view of the proximal end of a broach with which the invention can be used.

### BRIEF DESCRIPTION

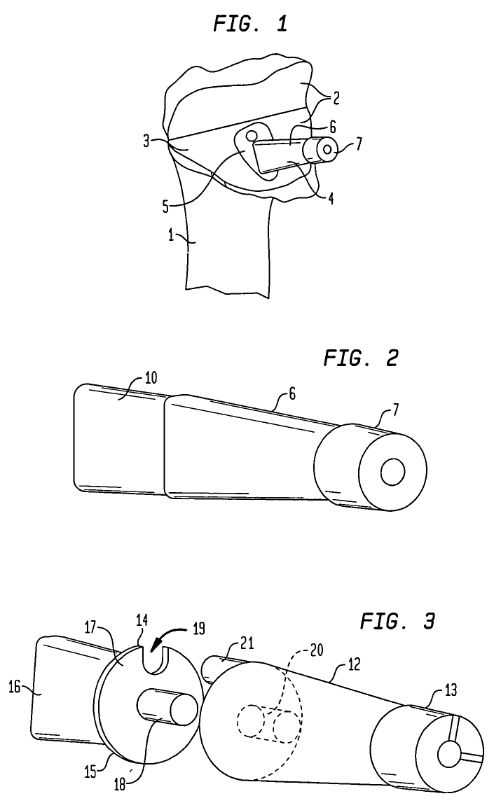

FIG. 1 is a diagrammatic illustration of the proximal end of a femur 1 which is resected at 2 to provide a flat planar surface 3 in which a femoral prosthesis 4 has been implanted. The prosthesis 4 is of modular construction and comprises a stem 5 located in the femur, a modular neck 6, the distal end of which is secured to the stem 5 and the proximal end of which is provided with a conical boss 7 which has a Morse taper and on which a spherical or part-spherical bearing head can be mounted.

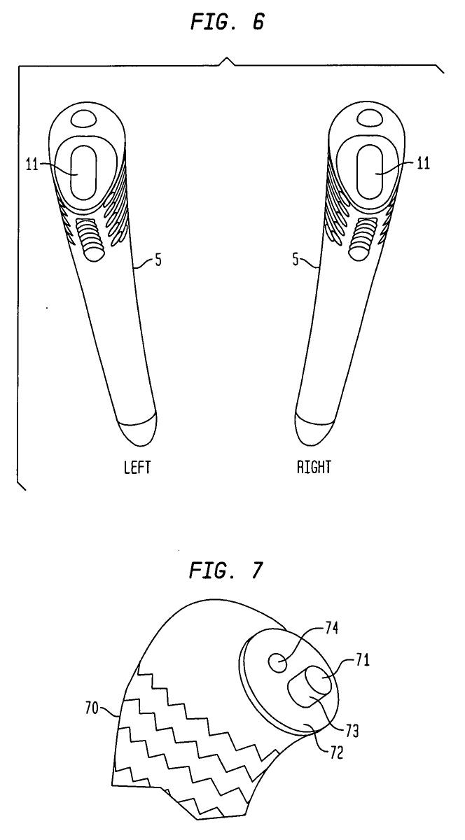

As will be seen from FIG. 2 the distal end of the modular neck 6 is provided with an engagement spade 10 which is shaped to locate in an elongated slot 11 which is most clearly shown in FIG. 6. The engagement spade 10 is dimensioned to be pushed or driven into the elongate by providing a tight or interference fit. Spade 10 may have curved ends connected by straight portions. As will be seen from FIG. 6 the modular femoral stem 5 can be made in left or right-handed versions.

FIG. 7 shows the proximal end of a broach 70 which can be used with the present invention and which has connection means 71 which include a flange 72 which has a central plug 75 and an angularly located socket 74 to receive a handle or other equipment.

Before fitting an implant of the type shown in FIG. 1 it is usual to provide a trial component to enable the surgeon to assess the necessary dimensions and consider whether there should be any degree of offset of the modular neck in relation to the stem.

The present invention, therefore, provides a trial prosthetic neck component which can be used with a trial femoral stem or broach, if suitable, or with an already implanted modular femoral stem. The trial prosthetic neck component is shown in FIG. 3 and comprises a shaped neck 12, the proximal end of which carries a tapered engagement boss 13 for a spherical bearing head (not shown) and the distal end of which has connection means 14 for removably connecting it to the distal end of the trial femoral stem or broach or the implanted stem. The trial stem can be basically similar to the stem 5 of the implanted prosthesis or it can be in the form of a broach as shown in FIG. 7 which has been used to define the opening in the bone. The broach is left in position and used as a trial stem. If a trial stem is used it is provided with an elongated slot similar to that shown in FIG. 6 to receive the trial neck. The connection means are therefore provided with a connector 15 which has a distal spade-shaped end 16 similar to the shape-shaped end 10 of the modular neck 6. The spade end extends from a flange 17 which has a central plug 18 and an angularly located socket 19. The distal end of the neck component 12 has a socket 20 to receive the plug 18 and a projecting abutment 21 to engage the angularly located socket 19. The connector can be made from a synthetic plastics material so that when it is assembled to the trial neck the plug 18 is a tight push fit into the socket 19 and the abutment 21 is a tight fit in the socket 19 so that the connector is firmly held in position. If the broach 7 is used for trials then the socket 20 and projecting abutment 21 can be used to engage the central plug 73 and socket 74, and the connected 15 is not required.

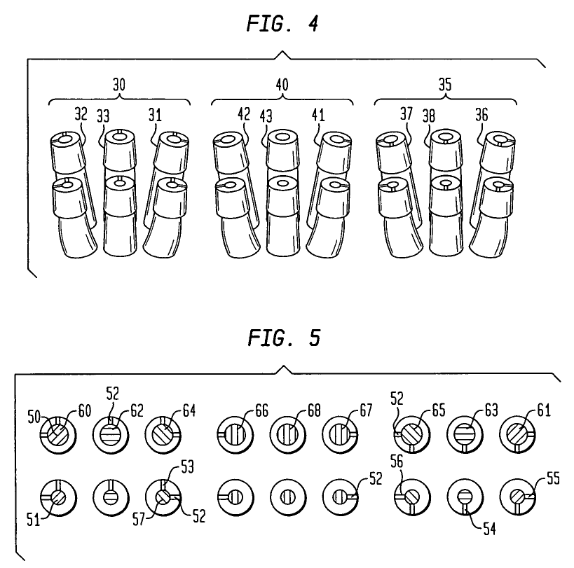

A number of trial necks are provided in different lengths and offsets so that the surgeon can select the trial neck which is appropriate for the patient. FIG. 4 illustrates eighteen modular trial necks of the kind shown in FIG. 3. In FIG. 4 the rear row of components all have long necks. The left hand group of three of these long-necked components and indicated by reference numeral 30 have valgus offset, one of which indicated by reference numeral 31 also has an anteverted offset and that indicated by reference numeral 32 has a retroverted offset and that indicated by reference numeral 33 has no anteverted or retroverted offset.

In the next group of neck components, indicated by reference numeral 35, in varus offset one of which indicated by reference numeral 36 has an anteverted offset, another, indicated by reference numeral 37, has a retroverted offset and the third, indicated by reference numeral 38

The remaining three of the long neck components and indicated by reference numeral 40 have no valgus or varus offset, one of the said three, indicated by reference numeral 41, has an anteverted offset, another indicated by reference numeral 42, has a retroverted offset and the third, indicated by reference numeral 43, has no anteverted or retroverted offset.

The trial neck components shown in the front row in FIG. 4 are similar to those shown in the back row and indicated by reference numerals 30 to 43 but all the front row components have a short neck.

The two lengths of neck and various offsets enable the surgeon to assemble the required length and offset but once he has selected the trial neck he requires he has to identify that neck with the modular necks which are to be used in the implant, and in order to assist the surgeon the proximal end faces of the trial necks carry indicea to indicate its length and any valgus, varus, anteverted, reverted offset.

FIG. 5 shows how the indicea is applied. The indicea indicating offset comprises a line extending in the direction of the offset. The indicea which indicates the length of the component is provided by a colored dot, the longer the length of the component the greater the diameter of the dot.

Each line indicating the offset extends outwardly from the length indicated dot which is provided as a central point on the proximal end of the neck component.

Inspection of FIG. 5 will show that the upper row of end surfaces all carry a large colored dot 50. All the lower row of end faces carry a small dot 51 to indicate that they are of the shorter length.

As will be seen the indicating lines 52 can extend in various directions according to their requirement, for example, a large central dot 50 indicates a long neck whereas a smaller dot (indicated by reference numeral 51) will indicate a short neck. No line associated to the dot indicates that the neck is neither varus nor valgus and neither anteverted nor retroverted. A vertical line, such as indicated by reference numeral 53, would indicate a valgus neck whereas a vertical line, as indicated by reference numeral 54, would indicate a varus neck. A horizontal line, such as indicated by reference numeral 55 indicates an anteverted neck and a horizontal line, as indicated by reference numeral 56, indicates a retroverted neck.

As a further example, a horizontal and vertical line such as shown at reference numeral 57 indicates a valgus neck which is anteverted.

Ten modular necks will be available to the surgeon, five of long length and five of short. As the spade stem 10 of the modular necks can be rotated through 180° only five of said necks of each length would be necessary. In order to identify the necks the colored dots and lines are of different colors. As shown in FIG. 5 those necks which have offsets which are the reciprocal of each other carry the same color. Thus, neck 60 with a large dot 50 carries the same color (yellow) as the head 61 which is its reciprocal when rotated through 180°. Head 62 is the reciprocal of head 63 and thus carries the same color (green). Head 64 is the reciprocal of head 65 and carries the same color (blue), head 66 is the reciprocal of head 67 and again carries the same color (brown). The remaining head which does not have any opposite (head 68) is color grey. The same color scheme is also applied to the short length necks.

When carrying out this surgery the surgeon first carries out various tests and the pre-operative planning of such a modular stem system requires three steps,
1) to first determine the centre of rotation of the acetabulum;
ii) to determine the metaphyseal position and sides of the femoral component;
iii) to determine the proper modular neck in terms of leg length and offset.

Other tests are carried out to decide on the choice of femoral component and the final choice of component size will depend on the intra-operative rotational stability of the broach.

The surgeon will first resect the femoral neck until the metaphyseal housing of the implant is prepared. A hollow chisel is preferably used which is adapted to the size of the implant and fixed to a broach handle. A cylinder of cancellous bone is removed from the metaphysis taking care not to damage the calcar of the greater trochanter. The smallest broach, left or right, is then introduced into the final medullary canal. Flexible reamers can be used up to the diameter corresponding to the chosen implant. After checking that they correspond to the side being operated broaches are fixed to the broach handle and they are introduced beginning with the smaller size up the size chosen during pre-op planning. The broach and and/or a trial prosthesis size will determine the size of the permanent implant. If the broach is unstable then a larger broach may be necessary.

A metal trial neck from the broach featuring the CCD angle and neck length chosen during pre-op templating, combined with a standard anteversion can be used first. The CCD angle on a neck length is selected by checking leg length and soft tissue tension and various head lengths can be used to enable fine tuning.

Once the most suitable CCD angle and neck head length have been determined the choice of the neck will be finally obtained by a good internal/external rotational ability in extension and a good stability of the hip in flexion/internal rotation/abduction and in extension external rotation/abduction.

In order to obtain these settings the trial neck components are used until the necessary position and length is found.

The trial neck and its connection are now removed together with the broach or trial stem and the modular stem which is coated with hydroxyapatite is introduced into the bone until it becomes wedged. The wedging can be completed by gentle hammering. The stem can have a small plastic stem holder at its proximal end to enable the surgeon to handle it without damage.

With the stem now in position further trials can be carried out again using the trial necks. To do so the trial neck which is selected needs to be attached to the connector which would be seated on the stem and the whole trial process will take place again, that is checking the CCD angle and length and checking the version.

With the correct trial neck now established the surgeon can take one of the ten modular heads supplied in the kit, the color and indicating lines showing which modular neck can be used. Once the implant is selected the insertion in the proper orientation is enabled with reference to the orientation of the colors and lines.

The modular stem is now inserted with its spade end in the elongated opening 11 and hammered into position. The bearing head (not shown) can now be fitted. The invention provides a test neck which, when used, indicates to the surgeon the precise type of modular neck which can be used to obtain the desired results. The proximal ends of the trial stems can also carry other information, for example size, numbers and any other indicea which may be of use to the surgeon.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A trial prosthetic hip system comprising:
a plurality of trial stems each having a recessed bore in a proximal end thereof; and,
at least three sets of trial necks each having a connection element for inserting into the bore in the trial stems, a first set of trial necks having a valgus offset with one of the necks of the first set being anteverted, one be retroverted and one being neutral in version, a second set of trial necks having a varus offset with one neck of the second set being anteverted, one being retroverted and one being neutral in version and a third set of trial necks being neutral in varus-valgus, one neck of the third set being anteverted, one being retroverted and one being anteverted and each trial neck of the first, second and third sets having a coded marking on a proximally facing surface indicating the varus/valgus, anteverted/retroverted and neutral orientations.

2. The trial prosthetic hip system as set forth in claim 1 wherein there are six sets of trial necks with three sets having short necks and three sets having long necks, the necks being coded to indicate which are short necks and which are long necks.

3. The trial prosthetic hip system as set forth in claim 2 wherein the neck lengths are indicated by colored circular dots having a diameter proportioned to the neck length.

4. The trial prosthetic hip system as set forth in claim 1 wherein the necks include a trunion for connection to a prosthetic femoral head, the trunion having a proximally facing surface including the varus/valgus, anteverted/retroverted and neutral orientation.

5. The trial prosthetic hip system as set forth in claim 4 wherein the offsets are indicated by lines extending along a proximally facing surface of the trunion.

6. The prosthetic hip as set forth in claim 1 wherein the recessed bore in the trial stem has a non-circular cross section and the connection element on the trial stem is an oval spade.

7. A trial prosthetic hip system comprising a plurality of trial stems each having a recessed bore in a proximal end thereof; and,
a plurality of sets of trial necks including sets having at least two different neck lengths and different varus/valgus and anteversion/retroversion orientations, each trial neck including a trunion for receiving a femoral head element, a proximally facing surface of the trunion including markings for indicating neck length, varus/valgus and anteversion/retroversion orientation.

8. The trial prosthetic hip system as set forth in claim 7 wherein there are six sets of trial necks with three sets having short necks and three sets having long necks, the necks being coded to indicate which are short necks and which are long necks.

9. The trial prosthetic hip system as set forth in claim 8 wherein the neck lengths are indicated by colored circular dots having a diameter proportioned to the neck length.

10. The trial prosthetic hip system as set forth in claim 7 wherein the offsets are indicated by lines extending along a proximally facing surface of the trunion.

11. The prosthetic hip as set forth in claim 7 wherein the recessed bore in the trial stem has a non-circular cross section and the connection element on the trial stem is an oval spade.

12. A trial prosthetic neck component adapted for use with a trial femoral implant and which has removable connection means at its distal end for connection to a trial medullary stem or broach or the actual stem, and removable connection means at its proximal end for connection to a bearing head, and in which said proximal end is provided with indicea to indicate its length and any valgus, varus, anteverted, retroverted offset.

13. The prosthetic neck component as claimed in claim 12 in which the indicea which indicates its length is provided by a colored dot the longer length the greater the diameter of the dot.

14. (canceled)

15. The prosthetic neck component as claimed in claim 13 in which each line indicating the offset extends outwardly from the length indicating circular dot provided at a central point on the proximal end of the component.

16. The prosthetic neck component as claimed in claim 12 in which the connecting means to the trial stem or broach or the actual stem includes a cylindrical plug and an angularly located socket.

17. The prosthetic neck component as claimed in claim 16 in which the cylindrical plug and angularly located socket are dimensioned to engage and fit an existing socket and projecting abutment on said broach and the connecting means also includes a connector the proximal end of which has a socket and projecting abutment to engage a plug and socket on the neck component and the distal end of which has a spade connector to engage and located in the trial stem or implanted femoral stem.

18. The prosthetic neck component as claimed in claim 12 which is provided in combination with a number of other similar neck components which have different lengths and/or offsets and which can be selectively connected to a trial medullary stem or broach and a bearing heads to provide a modular construction of trial femoral implants of different lengths and offsets.

19. The prosthetic neck component as claimed in claim 18 which is provided in combination with nineteen similar neck components, nine of the components having long necks, three of said long neck components having a valgus offset one of which also has an anteverted offset, another of which also has a retroverted offset, and the third of which has no anteverted or retroverted offset; three of said long neck components having a varus offset one of which also has an anteverted offset, another of which also has a retroverted offset and the third of which has no anteverted or retroverted offset,
and the remaining three of said long neck components having no varus or valgus offset, one of said three having an anteverted offset, another having a retroverted offset, and a third of which has no anteverted or retroverted offset;
the remaining nine neck components having short necks and a replicating the various offsets of the nine long neck components.

20. The prosthetic neck component as claimed in claim 19 in which each of the nine long neck trial components carries a colored dot to indicate the particular offset, each two components have offsets which are the reciprocal of each other and one of which has a valgus offset and the other a varus offset having the same color, and the three components which do not have a valgus or varus offset having the same color; and the other components having short necks and which do not have a valgus or varus offset having the same color; and the nine neck components having short necks and replicating the various offsets of the nine neck components having similar coding.
